(19) United States
(12) Patent Application Publication
(10) Pub. No.: US 2011/0196395 A1
(43) Pub. Date: Aug. 11, 2011

(54) Method for displaying an image of the inside of a vessel lying in front of an expander device and display device corresponding hereto

(76) Inventor: Michael Maschke, Lonnerstadt (DE)

(21) Appl. No.: 13/021,928

(22) Filed: Feb. 7, 2011

(30) Foreign Application Priority Data
Feb. 8, 2010 (DE) 10 2010 007 177.3

(51) Int. Cl. A61B 17/22

(52) U.S. Cl. 606/159

(57) ABSTRACT

A guide wire featuring a tip is introduced into a vessel. An expander tool featuring a tip is introduced by pushing it over the guide wire into the vessel. For the expander tool introduced into the vessel at least one image is output to the operator of the expander tool via a viewing device. The image is a representation of the area of the inside of the vessel which, seen in a local direction of advance of the expander tool, lies directly in front of the tip of the expander tool. The image is determined on the basis of at least one image dataset which was acquired by at least one sensor. The at least one sensor is arranged at the tip of the guide wire or on the expander tool behind its tip.

### CROSS REFERENCE TO RELATED APPLICATIONS

This application claims priority of German application No. 10 2010 007 177.3 filed Feb. 8, 2010, which is incorporated by reference herein in its entirety.

### FIELD OF THE INVENTION

The present invention relates to a method for displaying an image of an inside of a vessel lying in front of an expander device and a display device corresponding hereto.

### BACKGROUND OF THE INVENTION

The practice of undertaking a vessel dilation by means of a balloon catheter to combat and remove stenoses and similar vessel constrictions or even vessel stoppages is known. This procedure leads in the short term to a complete or almost complete rectification of the stenosis. In many cases however a stenosis occurs once again after a relatively short time with this type of treatment (so-called restenosis).

A known way of avoiding restenoses is to conduct an atherectomy instead of a vessel dilation. In the atherectomy a guide wire having a tip is first introduced into a vessel. Then an expander tool having a drilling, grinding or cutting tip is introduced into the vessel by pushing it over the guide wire into the vessel. The tip of the expander tool is able to be rotated by means of a hollow shaft surrounding the guide wire. If necessary material removed can be sucked out. As an example of such a prior art the reader is referred to US 2004/181249 A1 and U.S. Pat. No. 7,344,546 B2 purely by way of example.

The atherectomy is undertaken as a rule under X-ray control by means of contrast media. An image is thus output during the introduction of the expander tool into the vessel via a viewing device to an operator of the expander tool.

In the prior art procedure a side view of the vessel is presented. The disadvantage of this type of presentation is that only the vessel constriction itself (i.e. the remaining vessel cross section) is visible, but not the entire vessel including the plaque on the vessel wall, which in the final analysis causes the vessel constriction. In addition it is not possible to distinguish between vessel wall and plaque. In practice this means a significant risk, since there is especially the danger of perforating the vessel with the tip of the expander tool.

Presentation of the vessel wall without X-rays is known from US 2005/187571 A1. In this document it is only possible however to show the vessel wall to the side of the expander tool. Showing the area in front of the expander tool is not possible.

### SUMMARY OF THE INVENTION

The task of the present invention is to create options by means of which it is possible, with an expander tool introduced into the vessel, to be able to output to the operator of the expander tool via the display device a live image or an almost live image of the area of the inside of the vessel located immediately in front of the tip of the expander tool.

The object involving the method is achieved by a display method,
wherein a guide wire having a tip is introduced into a vessel,
wherein an expander tool having a tip is introduced into the vessel by being pushed over the guide wire,
wherein the image at the expander tool introduced into the vessel is output via a viewing device to an operator of the expander tool.

Inventively there is provision for designing a display method of the type mentioned at the start so that,
the image is a presentation of the area of the inside of the vessel which, seen in a local direction of advance of the expander tool, lies immediately in front of the tip of the expander tool, and
the image is determined on the basis of at least one image dataset which was detected by at least one sensor arranged at the tip of the guide wire or behind the tip of the expander tool on the expander tool.

In individual cases it can be possible to arrange a so-called forward-looking sensor (i.e. in the direction of advance of the expander tool) behind the tip of the expander tool and look through the rotating tip in a similar way to looking through a rotating aircraft propeller. As a rule the at least one sensor is however arranged at the tip of the—in this case hollow—guide wire. This makes it possible
for a sequence of image datasets to be recorded by the at least one sensor during the introduction of the guide wire into the vessel,
for each acquired image dataset to be assigned a length of advance of the tip of the guide wire,
for a length of advance of the tip of the expander tool introduced into the vessel to be recorded and
for it to be determined, on the basis of the acquired length of advance of the tip of the expander tool and the lengths of advance assigned to the image datasets, which of the image datasets of the sequence are to be included for determining the image.

The guide wire is not present in the image datasets acquired in this case. It is however possible for the location of the guide wire to be computationally deter mined in the image output to the operator of the expander tool and then marked.

The type and number of the images output can vary if necessary. It is preferred for the image output (or in the case of a number of output images, at least one of the images) to be a perspective presentation which starts from the point of view lying inside the vessel and from there covers a spatial angle. The local direction of advance preferably lies in this case in approximately the center of the spatial angle covered.

The point of view in this case once again lies, seen in the local direction of advance of the expander tool, in the area of the tip of the expander tool.

The sensor can be embodied as required. In particular the at least one sensor can be embodied as an optical sensor, as an ultrasound sensor or as a magnetic resonance sensor. Combinations of these types of sensor are also possible. For example two sensors can be present of which one is embodied as an optical sensor and one as an ultrasound sensor respectively. The image in this case can be a combination image in which image datasets acquired by means of the ultrasound sensor and also by means of the optical sensor are included. As an alternative a separate respective image can be determined on the basis of the optically acquired image datasets and the image datasets acquired by means of ultrasound.

In a preferred embodiment of the inventive display method there is provision for the expected location of the expander tool on further introduction of the expander tool to be marked in the image output to the operator of the expander tool. This procedure makes better handling of the expander tool by the operator possible.

The object is also obtained in terms the device by a display device,
wherein the display device has an introduction device,
wherein the introduction devices has a guide wire which has a tip and is able to be introduced into a vessel,
wherein the introduction devices features an expander device having a tip and able to be introduced into the vessel by pushing it over the guide wire,
wherein the display device has a viewing device via which the image is able to be output to an operator of the expander tool.

Inventively there is provision for embodying a display device of the type described at the start by
arranging at least one sensor at the tip of the guide wire or behind the tip of the expander tool, by means of which an image dataset is able to be acquired at an introduction device introduced into the vessel,
the display device featuring at least one processing device which is connected for accepting the image dataset with the at least one sensor, from which on the basis of the image dataset the image is able to be determined and which is connected for output of the image to the display device, and
the at least one sensor and the processing device cooperating in operation of the display device such that the image output via the display device is a presentation of the area of the inside of the vessel which, seen in the local direction of advance of the expander tool, lies immediately in front of the tip of the expander tool.

Advantageous embodiments of the display device are possible. The advantageous embodiments of the display device essentially correspond to the advantageous embodiments of the inventive display method described above.

### BRIEF DESCRIPTION OF THE DRAWINGS

Further advantages and details emerge from the subsequent description of exemplary embodiments in conjunction with the drawings. The figures show the following basic diagrams:

FIG. 1 a display device,

FIGS. 2 and 3 a schematic diagram of a vessel with a guide wire,

FIG. 4 a schematic diagram of a vessel with a guide wire and an expander tool,

FIG. 5 a side view through the vessel,

FIG. 6 an image output to an operator of the expander tool,

FIG. 7 the front part of a guide wire,

FIG. 8 a possible cross section through the guide wire of FIG. 7 and

FIG. 9 a further possible cross-section through the guide wire of FIG. 7.

### DETAILED DESCRIPTION OF THE INVENTION

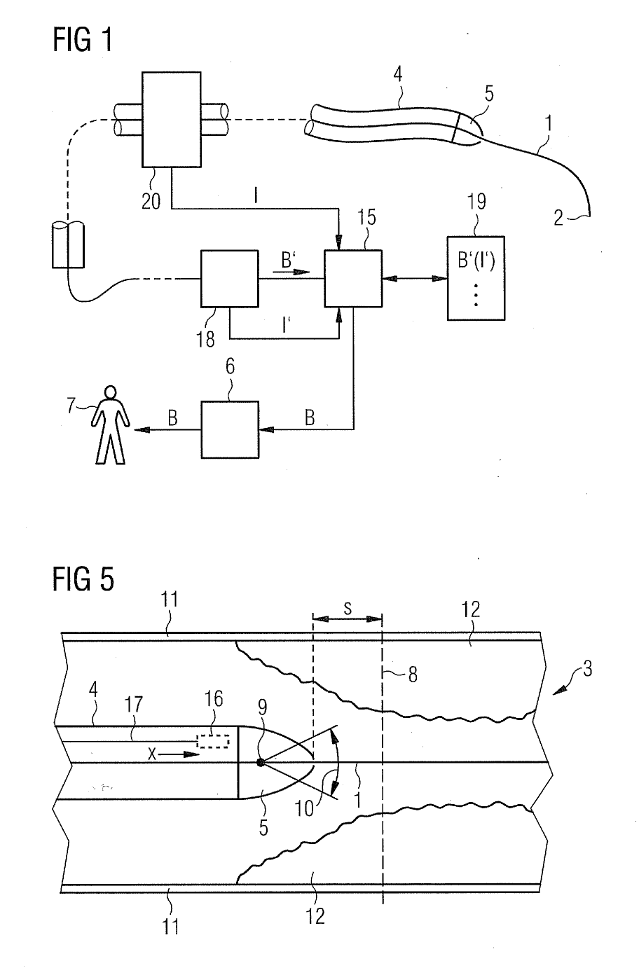

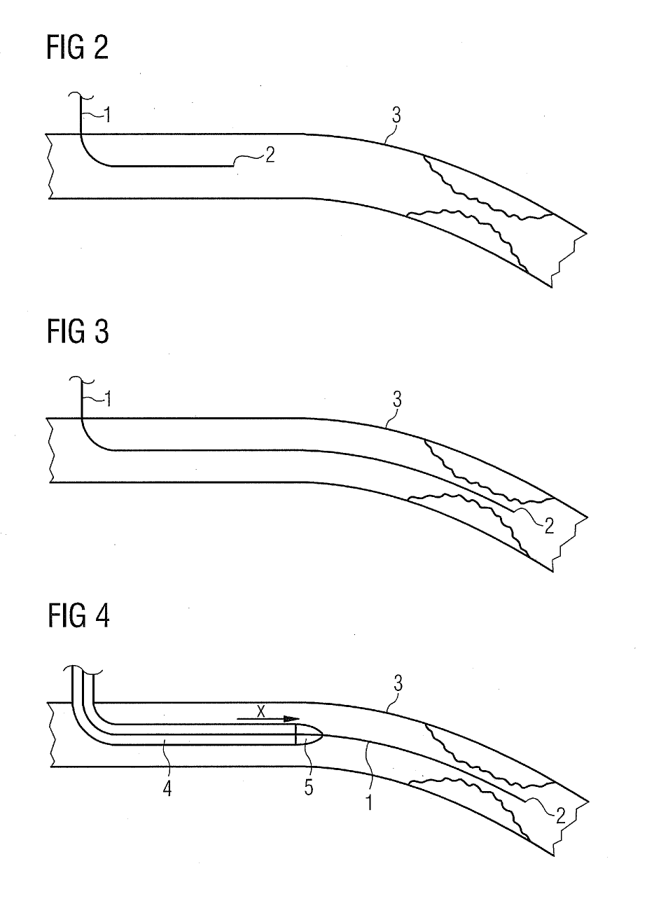

In accordance with FIG. 1 a display device features a guide wire 1. The guide wire 1 can have a significant length. In particular the guide wire 1 can have a length which is greater than 1 m, for example 2 m or 3.5 m. The guide wire 1 has a tip 2. Beginning with its tip 2, the guide wire is gradually introduced into the vessel 3, in accordance with the schematic diagram shown in FIGS. 2 and 3.

The display device also features an expander tool 4. The expander tool 4 and the guide wire 1 together form an introduction device of the display device.

The expander tool 4 has a tip 5. The tip 5 of the expander tool 4 is—at least temporarily—driven rotationally by a hollow shaft. The tip 5 of the expander tool 4 serves to remove material on the inside of the vessel 3 as it rotates. The tip 5 of the expander tool 4 can for example be embodied as a drill, grinding or cutting tip for this purpose.

The expander tool 4 is likewise introduced into the vessel 3, beginning with its tip 5. The tool is introduced into the vessel 3 in accordance with the schematic diagram depicted in FIG. 4 by the expander tool 4 being pushed over the guide wire 1.

The display device further features a viewing device 6. An image B is output to an operator 7 of the expander tool 4 via the viewing device 6 during the introduction (=process) of the expander tool 4 into the vessel 3 and above all also for the expander tool 4 introduced into the vessel (=state).

For the sake of neatness it should be mentioned that introduction (=process) and having been introduced (=state) are not mutually exclusive. During introduction (=process) it matters however on that the guide wire 1 or the expander tool 4 are moved, i.e. the location of the tip 2 of the guide wire 1 or the tip 5 of the expander tool 4 is thus not constant. After introduction (=state) it matters on the other hand that the expander tool 4 the guide wire 1 are located in the vessel 3, i.e. are introduced.

Where previously described, the display device and its operation are known from the prior art. In particular the introduction device, i.e. the unit consisting of guide wire 1 an expander tool 4, is known from US 2004/181249 A1 and U.S. Pat. No. 7,344,546 B2.

To make it easier for the operator 7 of the expander tool 4 to decide when and if necessary in which direction of rotation the tip 5 of the expander tool 4 is to be rotated, the image B output via the viewing device 6 is a representation of an area of the inside of the vessel 3. The area shown lies in accordance with FIG. 5, seen in a direction of advance x of the expander tool 4, immediately in front of the tip 5 of the expander tool 4.

The direction of advance x of the expander tool 4 is determined by the course of the guide wire 1. The guide wire 1 for its part follows the course of the vessel 3. The direction of advance x thus varies from location to location, in accordance with the course of the vessel 3. For this reason the direction of advance x is referred to below as the local direction of advance x.

The image B output to the operator 7 can typically involve a presentation of the vessel 3 in cross-section, with a cut line 8 along which the cross-section is shown lying in accordance with the diagram shown in FIG. 5 at a predetermined distance s in front of the tip 5 of the expander tool 4. Preferably the image B however involves a perspective presentation which, in accordance with FIG. 5, starts from one point of view 9 and from there covers a spatial angle 10. The point of view 9 lies within the vessel 3, preferably in the area of the tip 5 of the expander tool 4, shortly in front of it or shortly behind it. The spatial angle 10 is preferably determined such that the local direction of advance x lies approximately in the center of the spatial angle 10 covered.

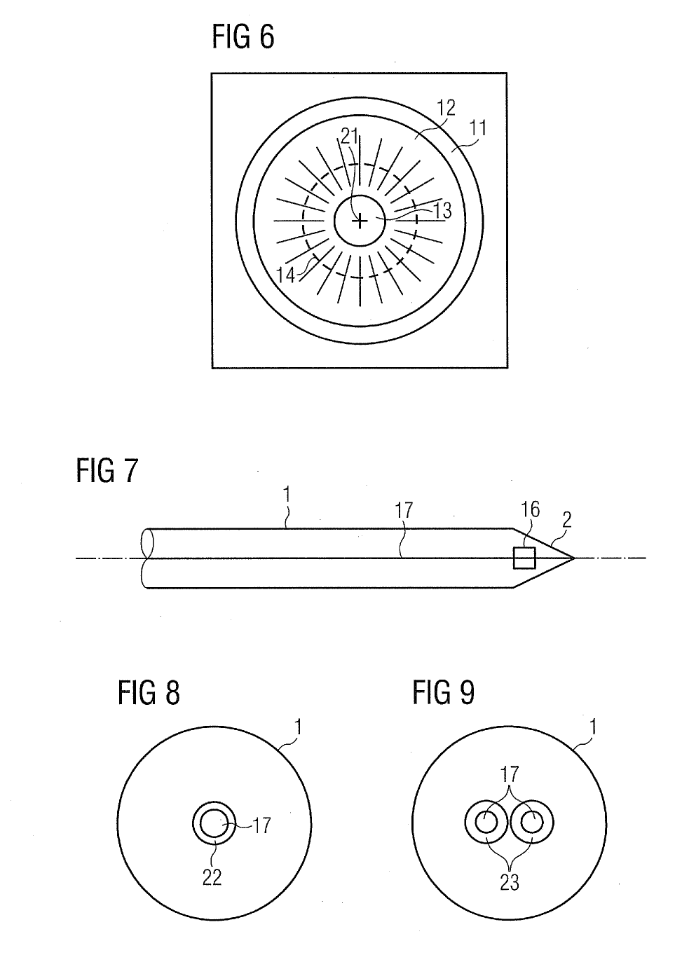

FIG. 6 shows an example of a perspective diagram, as has been explained above. In FIG. 6 the vessel wall 11 can initially be seen. An area 12 covered by plaque is also visible in FIG. 6. Thus only a residual opening 13 of the vessel 3 remains for the blood flow.

In the image B output to the operator 7 of the expander tool 4 the location at which the expander tool 4 is expected is marked in accordance with FIG. 6 if the expander tool is introduced further into the vessel 3, i.e. typically the tip 5 of the expander tool 4 is moved forwards up to the cut line (see FIG. 5). A corresponding marking is provided in FIG. 6 with the reference number 14. If the effective diameter of the expander tool 4 can vary—is dependent on the direction of rotation for example—a number of corresponding markings can also be indicated accordingly in the image B output to the operator 7 of the expander tool 4.

The image B output to the operator 7 of the expander tool 4 is determined by a processing device 15 which is connected in accordance with FIG. 1 for output of the image B to the viewing device 6. The image B is determined by the processing device 15 on the basis of at least one image dataset B′ which is acquired by at least one sensor 16. The processing device 15 is connected in accordance with FIG. 1 to the at least one sensor 16 for accepting the at least one image dataset B′.

In individual cases it can be possible to arrange the at least one sensor 16 in the expander tool 4. In this case the at least one sensor 16 is generally arranged in the expander tool 4 behind the rotatable tip 5 of the expander tool 4. A “direction of view” of the at least one sensor points forwards in this case. The at least one sensor 16 in this case detects the image dataset B′ at a point in time at which the expander tool 4 is already introduced into the vessel 3. Indicated by a dashed-line rectangle in FIG. 5 is a possible positioning of the at least one sensor 16. The at least one sensor 16, if it is arranged in the expander tool 4, is connected by means of a signal line 17 routed in the expander tool 4 to the processing device 15. The image dataset B′ acquired by the at least one sensor can in this case—after processing by the processing device 15—be output directly as image B via the viewing device 6 to the operator 7 of the expander tool 4.

The image B output via the viewing device 6 to the operator 7 of the expander tool 4 is an image acquired live in this case. The determination of the image B by the processing device 15 is reduced in this case to a simple processing of the acquired image dataset B′.

The at least one sensor 16 can also be arranged in accordance with FIG. 7 at the tip 2 of the guide wire 1. If it is not possible to arrange the at least one sensor 16 in the expander tool 4, the arrangement of the at least one sensor 16 at the tip 2 of the guide wire 1 is realized as an alternative to an arrangement in the expander tool 4. Otherwise it is realized as an alternative or cumulatively to an arrangement on the expander tool 4.

In the case of the arrangement of the at least one sensor 16 at the tip 2 of the guide wire 1, the process is as follows:

During the introduction of the guide wire 1 into the vessel 3 a new image dataset B′ is repeatedly acquired by means of a sensor 16, i.e. overall a sequence of image datasets B′. The at least one sensor 16 feeds the image datasets B′ that it has acquired to the processing device 15. The processing device 15 accepts the image datasets B′. The connection between the at least one sensor 16 and the processing device 15 can for example be made using a corresponding signal line 17 which runs inside the guide wire 1—hollow for this purpose.

The processing device 15 continues to accept for each image dataset B′ a respective length of advance l′ by which the tip 2 of the guide wire 1 was inserted into the vessel 3 at the point that the corresponding image dataset B′ was acquired. The length of advance l′ of the tip 2 of the guide wire 1 can for example be acquired by means of a corresponding detection device 18 and fed from there to the processing device 15.

The processing device 15 allocates each acquired image dataset B′ to the corresponding length of advance l′ and stores the respective image dataset B′ including the assigned length of advance l′ in a storage device 19.

On introduction of the expander tool 4 into the vessel 3 the processing device 15 accepts a length of advance l of the tip 5 of the expander tool 4. The length of advance l of the tip 5 of the expander tool 4 can for example be acquired by means of a corresponding detection device 20 and fed from this device to the processing device 15.

The processing device 15 determines the image B to be output via the viewing device 6 by initially determining, on the basis of the length of advance l of the tip 5 of the expander tool 4 and the lengths of advance l′ of the tip 2 of the guide wire 1 allocated to the stored image, which of the stored image datasets B′ is to be included for determining the image B. Then it determines the image B on the basis of the corresponding image datasets B′ determined.

If for example each sensor 16 arranged at the tip 2 of the guide wire 1 has already acquired an image dataset B′ itself which corresponds to a perspective presentation similar to the presentation of FIG. 6, in the simplest case that image dataset B′ can be selected of which the assigned length of advance l′ comes closest to the current length of advance l of the tip 5 of the expander tool 4. If necessary this selection can be restricted to those image datasets B′ of which the assigned length of advance l′ is greater than the length of advance l of the tip 5 of the expander tool 4.

Other procedures are also possible, for example an interpolation of the number of stored image datasets B′ or determining in some other way the image B to be output. Independently of the actual embodiment of the display device, the at least one sensor 16 and the processing device 15 interact during operation such that the image B output via the viewing device 6 is a presentation of the area of the inside of the vessel 3 which, seen in the local direction of advance x of the expander tool 4, lies directly in front of the tip 5 of the expander tool 4. This applies both in the case in which the at least one sensor 16 is arranged in the expander tool 4 and also in the case in which the at least one sensor 16 is arranged in the guide wire 1.

If the at least one sensor 16 is arranged in the tip 2 of the guide wire 1, the acquired image datasets B′ do not contain the guide wire 1 itself. It is however possible for the processing device 15 to determine the location of the guide wire 1 in the image B output to the operator 7 of the expander tool 4 and to insert a corresponding marking 21 (see FIG. 6) into the image B.

The at least one sensor 16 can—regardless of the position at which it is arranged—be embodied as required. In particular the at least one sensor 16 can be embodied as an optical sensor, as an ultrasound sensor or as a magnetic resonance sensor. Combinations of the individual sensor types with one another are also possible. Thus for example, in accordance with the diagram shown in FIG. 7, two sensors 16 can be present of which one is embodied as an optical sensor and one as an ultrasound sensor respectively.

The cross-section of the guide wire 1 can be embodied depending on the position of the signals to be transmitted via the guide wire 1 from the sensors 16 to the processing device 15. If only a single sensor 16 is arranged at the tip 2 of the guide wire 1, it is generally sufficient for the guide wire 1, in accordance with FIG. 8, to have an essentially circular central cutout 22 in which the signal line 17 is routed. If a number of sensors 16 are present, the central cutout 22 can be dimensioned larger, so that all signal lines 17 can be routed in the central cutout 22. As an alternative it is possible, in accordance with FIG. 9, for the guide wire 1 to have a separate guide channel 23 in each case for each signal line 17.

The present invention offers significant advantages compared to the prior art. In particular it is possible, during operation of the expander tool, to allow the operator 7 to look forwards so that the operator 7 sees where he is working with the tip 5 of the expander tool 4.

The above description serves exclusively to explain the present invention. The scope of the protection of the present invention on the other hand is intended to be exclusively defined by the enclosed claims.

### LIST OF REFERENCE CHARACTERS

1 Guide wire
2 Tip of the guide wire
3 Vessel
4 Expander tool
5 Tip of the expander tool
6 Viewing device
7 Operator
8 Cut line
9 Point of view
10 Spatial angle
11 Vessel wall
12 Area
13 Residual opening
14, 21 Markings
15 Processing device
16 Sensors
17 Signal lines
18, 20 Detection devices
19 Storage device
22 Center cutout
23 Guide channels
B Image
B′ Image datasets
l, l′ Lengths of advance
s Distance
x Direction of advance

1.-12. (canceled)

13. A method for displaying an image, comprising:
introducing a guide wire having a tip into a vessel;
introducing an expander tool having a tip into the vessel by pushing the expander tool over the guide wire;
acquiring an image dataset of an inside of a vessel in a local direction of advance of the expander tool and in front of the tip of the expander tool by a sensor;
determining an image based on the image dataset by a processing device; and
displaying the image to an operator of the expander tool by a viewing device.

14. The method as claimed in claim 13, further comprising:
arranging the sensor at the tip of the guide wire,
acquiring a sequence of image datasets by the sensor while introducing the guide wire into the vessel, the image datasets being assigned a sequence of lengths of advance of the tip of the guide wire,
acquiring a length of advance of the tip of the expander tool introduced into the vessel, and
determining the image based on the image datasets, the acquired length of advance of the tip of the expander tool, and the lengths of advance of the tip of the guide wire assigned to the image datasets.

15. The method as claimed in claim 13, wherein a location of the guide wire is marked in the image output to the operator of the expander tool.

16. The method as claimed in claim 13, wherein the image is a perspective representation covering a spatial angle within the vessel and the local direction of advance lies in a center of the spatial angle.

17. The method as claimed in claim 13, wherein the sensor is an optical sensor, an ultrasound sensor, or a magnetic resonance sensor.

18. The method as claimed in claim 13, wherein a location of the expander tool is marked in the image output to the operator of the expander tool

19. The method as claimed in claim 13, wherein the sensor is arranged on the expander tool and behind the tip of the expander tool.

20. A display device for displaying an image, comprising:
an introduction device comprising:
a guide wire having a tip to be introduced into a vessel, and
an expander tool having a tip to be introduced into the vessel by pushing the expender tool over the guide wire;
a sensor for acquiring an image dataset of an inside of the vessel in a local direction of advance of the expander tool and directly in front of the tip of the expander tool while introducing the instruction device into the vessel;
a processing device for receiving the image dataset and for determining an image based on the image dataset; and
a viewing device for displaying the image to an operator of the expander tool.

21. The display device as claimed in claim 20, wherein:
the sensor is arranged at the tip of the guide wire and acquires a sequence of image datasets whiling introducing the guide wire into the vessel,
the processing device assigns a sequence of lengths of advance of the tip of the guide wire to the image datasets and stores the image datasets and the lengths of advance of the tip of the guide wire,
the processing device is supplied with a length of advance of the tip of the expander tool introduced into the vessel, and
the processing device deter mines the image based on the image data sets, the acquired length of advance of the tip of the expander tool, the lengths of advance the tip of the guide wire assigned to the image datasets.

22. The display device as claimed in claim 20, wherein the processing device marks a location of the guide wire in the image output to the operator of the expander tool.

23. The display device as claimed in claim 20, wherein the image is a perspective representation covering a spatial angle within the vessel and the local direction of advance lies in a center of the spatial angle.

24. The display device as claimed in claim 20, wherein the sensor is an optical sensor, an ultrasound sensor, or a magnetic resonance sensor.

25. The display device as claimed in claim 20, wherein the processing device marks a location of the expander tool in the image output to the operator of the expander tool.

26. The display device as claimed in claim 20, wherein the sensor is arranged on the expander tool and behind the tip of the expander tool.
